(19) United States
(12) Patent Application Publication
(10) Pub. No.: US 2012/0040391 A1
(43) Pub. Date: Feb. 16, 2012

(54) Sepsis diagnostic test

(75) Inventors: Steffen Mitzner, Rostock (DE); Jens Altrichter, Kavelstorf (DE); Hans-Joachim Dollmantel, Rostock (DE); Jana Sulz, Rostock (DE)

(73) Assignee: GAMBRO LUNIDA AB, LUND (SE)

(21) Appl. No.: 11/631,014

(22) PCT Filed: Jun. 23, 2005
(86) PCT No.: PCT/EP05/52950

§ 371 Date: Jun. 15, 2009

Related U.S. Application Data
(60) Provisional application No. 60/585,091, filed on Jul. 6, 2004.

(30) Foreign Application Priority Data
Jul. 2, 2004 (PA) 200401051

(51) Int. Cl. C12Q 1/02

(52) U.S. Cl. 435/29

(57) ABSTRACT

The invention relates to a method for the extracorporeal qualitative or semi-quantitative determination of the amount of indicators for the systemic inflammatory response system (SIRS) or sepsis in the blood, blood serum, blood plasma, other body fluids or lavages or their constituents of human or animal subjects. In order to provide a test which is better than that of the prior art, with which the presence and/or the severity of SIRS or sepsis can be rapidly, economically, reliably and reproducibly determined in a sample, such as blood serum of a patient. To this end, the inventive method has the following steps in which: a cell line is prepared in a culture; in at least one first preparation, cells of the cell line are brought into contact with blood, blood serum or blood plasma, other body fluids or lavages or their constituents of a human or animal subject to be examined, and into contact with a reagent that reacts to reactive oxygen intermediates (ROI), and enters into a color, light or other measurable reaction; in at least one other preparation, cells of the cell line are brought into contact with blood, blood serum or blood plasma, other body fluids or lavages or their constituents of a human or animal control subject that is not ill with SIRS or sepsis, and into contact with a reagent that reacts to reactive oxygen intermediates (ROI), and enters into a color, light or other measurable reaction; the color, light or other measurable reactions are measured in the preparations, and; the measured values of the subject to be examined are compared with those of the control subject.

### SUBJECT-MATTER OF THE INVENTION

The invention relates to a method for the extracorporeal qualitative or semi-quantitative determination of the quantity of indicators for SIRS or sepsis in the blood, blood serum, blood plasma, other body fluids or lavages or constituents thereof of human or animal subjects. The invention also relates to a corresponding test kit which contains the constituents necessary to carry out the method according to the invention.

### BACKGROUND TO THE INVENTION

The pathogenesis of SIRS (Systemic Inflammatory Response System) and sepsis up to the prelethal stages of septic shock and multiple organ failure is substantially attributed to a dysfunction of the immune system (Grimminger F et al. 1997). A reliable prognosis of the course and successful immunomodulatory therapy of SIRS/sepsis require a well defined stage classification and diagnostic methods suitable for the purpose.

At present, only rough outlines can be produced when distinguishing a hyperinflammatory from an anti-inflammatory phase, using the course kinetics of some cytokines (Grimminger F et al. 1997. Romaschin A D et al. 1998). Because of the high variability in the cytokine level of the patients and the complex network of relationships between the individual cytokines, no practical use for diagnosis and therapy has resulted as yet in this field. Other inflammation markers such as CRP and procalcitonin (Oberhoffer M et al. 1999, Mimoz O et al. 1998, Al-Nawas B et al. 1995/96) have a relatively high predictive value for the outcome or the disease, but give no indications whatever for an immunomodulatory treatment.

At the cellular level of the immune system, an important role is ascribed to the monocycles and (neutrophilic) granulocytes in the course of SIRS/sepsis (Nussler A K et al. 1999). Monocytes and cells related to them (e.g. macrophages, dentritic cells) initiate the immune defence by antigen presentation and spilllage of lymphocyte-stimulating cytokines. The extent of the HLA-DR expression on circulating monocytes is used as a parameter for the prognosis of the risk of sepsis in trauma and SIRS patients and the severity of the course of the disease once sepsis has set in (lethality risk) (Wakefield C H et al. 1993, Asadullahn K et al. 1995). This HLA-DR diagnosis can also be coupled with an immunomodulatory therapy. A fatal course of the disease can be prevented with an administration of specific cytokines (IFN-G, G-CSF), the dosage of which is geared to the increase in the HLA-DR expression (Döcke W D et al. 1997).

Moreover, activated monocycles (macrophages) are capable together with the (neutrophilic) granulocytes of the direct elimination of causative agents by phagocytosis (eater cells) and the spillage of reactive oxygen intermediates (ROI) and nitrogen oxides. Due to chemotactic stimuli, both cell types can leave the circulation and develop their microbicidal activity following tissue infiltration.

These elemental defence functions of the cells depend both on endogenous factors, such as e.g. the degree of maturity of the cells, and to an even greater extent on exogenous factors, such as cytokines, chemokines, metabolites, endotoxins and other causative agent products, Because of these complex relationships, the investigation of these leukocycle functions during the course of SRIS/sepsis faces major difficulties as regards method.

Phagocytosis activity and ROI production (oxyburst) are as a rule determined only in the case of peripheral blood leukocytes, as the diagnostically more interesting resident tissue leukocytes are, with a few exceptions (e.g. peritoneal macrophages), scarcely accessible for these determinations (Dong Y L et al. 1993, Holzer K et al. 2000). In addition, phagocytosis and oxyburst are triggered only by the addition of “standard” stimulants (bacteria or microparticles, f-MLP, PMA) (EP 0 435 226, DE 41 17 459), the effect of which can cover specifically disease-induced modulators of leukocyte activity in patient plasma.

The reverse route, the investigation of the influence of patient plasma and factors therefrom on the activity of healthy control leukocytes, fails due to the changed characteristics of isolated leukocytes separated from autologous plasma (Pascual C et al. 1997) and the difficulties of standardization because of the heterogeneity of such populations.

### OBJECT OF THE INVENTION

The object of the invention was to provide a test, improved compared with the state of the art, in which the presence and/or the severity of SIRS or sepsis can be quickly, cheaply, reliably and reproducibly established in a sample, such as e.g. blood serum of a patient.

### DESCRIPTION OF THE INVENTION

The object forming the basis of the invention is achieved by a method for the extracorporeal qualitative or semi-quantitative determination of the quantity of indicators for SIRS or sepsis in the blood, blood serum, blood plasma, other body fluids or lavages or constituents thereof of human or animal subjects, wherein the method comprises the steps in which
a cell line is prepared in culture,
in at least one first preparation, cells of the cell line are brought into contact with blood, blood serum or blood plasma, other body fluids or lavages or constituents thereof of a human or animal subject to be examined, and with a reagent which responds to oxygen intermediates (ROI) and enters into a colour, light or other measurable reaction,
in at least one further preparation, cells of the cell line are brought into contact with blood, blood serum or blood plasma, other body fluids or lavages or constituents thereof of a human or animal control subject not ill with SIRS or sepsis, and with a reagent which responds to oxygen intermediates (ROI) and enters into a colour, light or other measurable reaction,
the colour, light or other measurable reactions in the preparations are measured and the measured values of the subject to be examined are compared with those of the control subject.

With the method according to the invention, instead of native leukocytes, permanent cell lines are used which, despite an unlimited partition capacity, have retained essential leukocyte functions (phagocytosis, ROI production, spillage of cytokines, chemokines). Within the population, the cell lines used according to the invention have a uniform reactivity that remains constant over many generations, which is why their use as sensor cells in the test method according to the invention delivers considerably better reproducible and comparable results than for example native leukocytes.

In an embodiment quite particularly preferred according to the invention, the cell line used is therefore a leukocytary cell line or leukocytes cell line.

Essential within the meaning of the invention is that the permanent cell lines are spontaneously excited, after suitable differentiation steps by the application of a sample, such as e.g. human serum, to produce ROI. Additionally, triggering agents such as microparticles, N-fMLP or PMA, such as are customary in the case of oxyburst measurements on native leukocytes, are not used.

According to the invention, cell lines which are used in this case are those which respond to indicators for the presence or absence of indicators for SIRS or sepsis in a sample accompanied by the release of oxygen intermediates (ROI), wherein the quantity of the released oxygen intermediates (ROI) correlates with the concentration of indicators for SIRS or sepsis in a sample.

Suitable cell lines comprise for example lines established by hybridization of native monocytes or macrophages with suitable tumor cells followed by cloning, such as are described in U.S. Pat. No. 4,737,455. Other suitable cell lines can be attributed to isolates from spontaneously formed or induced tumors (e.g. leukemias), wherein some cells still have to be differentiated out by additional induction steps to phagocytizing and ROI producing cells. Among these cell types, a dominant role falls to the human promyelocytic HL-60 line (Breitman T R et al. 1980, Collins S J 1987). Limitlessly multipliable in simple culture media with the stable-remaining characteristic of precursor cells, HL-60 cells can be differentiated out by specific inductors into granulocytic (e.g. by all-trans-vitamin A acid) or monocyctic (e.g. by vitamin D3 derivatives). After induction of the terminal differentiation, these cells are still capable of partition for only a limited time and end in a homogeneous population which in many properties is the same as native granulocytes or monocytes. Their ease of handling and ability to be influenced predestines the HL-60 line for use as sensor cells in the clinical and pharmaceutical sectors. Cell lines preferred according to the invention are HL-60 (ATCC, CCL-240), THP-1 (ATCC, TIP-202 or DSMZ, ACC 16) and U937 (ATCC, CRL-1593.2 or DSMZ, ACC 5).

Checks applied hitherto to the leukocyte functions phagocytosis and ROI production in the whole blood of a patient or in plasma after erythrocyte sedimentation, which take place with throughflow cytometry, fluorometry, gluorescence miscoscopy and chemiluminescence measurement, do not differentiate in the case of sepsis patients between the given ability of the leukocytes to function and the immunomodulatory influences of plasma factors. In particular in the case of fluorometry and chemiluminescence measurement, an additional variability results due to the fluctuations in the respective leukocyte count. The use according to the invention of established cell lines that are also well investigated in respect of their properties offers, on the other hand, a test which is very largely independent of the fluctuations in respect to the ability of the sensor cells to function.

Moreover, when using purified leukocytes in such a test there is the danger of the preactivation of still quiescent bis-N cells during the cleaning process, as a result of which the reproducibility and reliability of the measurements are clearly impaired. Also, for a laboratory engaged in routine work, working with purified leukocytes means a high outlay in terms of method and time which can be clearly reduced by the method of the present invention and the provision of a corresponding test kit.

In one respect, therefore, when determining the quantity of indicators for SIRS or sepsis in body fluids, preferably in the blood, the present invention replaces isolates of native leukocytes with reproducibly obtainable and easily standardizable biosensors.

The formed oxygen intermediates (ROI) are detected via a measurable reaction, preferably a light reaction. Detection is quite particularly preferably via a chemiluminescence reaction. Particularly preferred reagents for the chemiluminescence reaction are bis-N-methylacridiniumnitrate (lucigenin) and 5-amino-1,2,3,4-tetrahydrophthalazine-1,2-dione (luminol) which can be used on their own or also mixed. Lucigenin is a quite particularly preferred reagent.

Chemiluminescence is expediently measured by means of a luminometer. With such a measurement, a kinesis is suitably carried out over a period of preferably 2-120 min.

Surprisingly, it was found that the chemoluminscence triggered with the method according to the invention showed deviations from the values of healthy subjects in the case of serum samples from a collective of SIRS or sepsis patients. As the severity of the course of the disease increases, the chemiluminscence caused by the serum sample falls below the normal range which is ascertained in healthy subjects. The more serious the symptoms of a SIRS or sepsis patient, the smaller is thus the quantity of the oxygen intermediates (ROI) formed in the method according to the invention with a patient sample and thus also the measured reaction, e.g chemoluminscence, with the reagent used. The severity of the disease and the lethality risk of SIRS or sepsis patients can already be recognized by the method according to the invention on the day of admission from the decreased reaction with the reagent used, such as e.g. decreased chemiluminescence values. In every case a comparison with the corresponding values of one or more control samples of healthy patients is necessary.

Time-consuming scoring systems or laborious and costly determinations such as ELISAs and FACS analyses can therefore be replaced by a relatively simple and cheap test. Based on an ensured correlation with SIRS sepsis courses, the described test method can deliver a rapidly and easily ascertainable indication for therapeutic measures.

In a further preferred embodiment of the method according to the invention, the measurable reaction used to detect the formed oxygen intermediates (ROI) is a fluorescence reaction. Reagents suitable for this are in particular dihydro-rhodamine and hydroethidium or a mixture of the two. In this case the fluorescence reaction is expediently measured by means of fluorometry or throughflow cytometry. An advantage of this is that the intracellular fluorescence that takes place inside the sensor cells with reagent that has penetrated into the cells can also be measured by means of fluorometry and throughflow cytometry.

In a further preferred embodiment of the method according to the invention, the measurable reaction used to detect the formed oxygen intermediates (ROI) is a colour reaction. Reagents suitable for this are in particular iron-III-cytochrome, nitrotetrazolium or a benzidine derivative, preferably 3,3′-5,5′-tetramethylbenzidine, or a mixture of at least two of the aforementioned. In this case the colour reaction is expediently measured by means of a spectral photometer.

It is particularly expedient according to the invention if, before being brought into contact with the blood, blood plasma or blood serum, other body fluids or lavages or constituents thereof, the cells of the cell line are treated in culture for a period of time with inductors of the leukocyte differentiation, preferably with interferon gamma (IFN) and/or 1-alpha-25-dihydroxycholecalciferol (VD3) and/or granulocyte colony-stimulating factor (G-CSF) and/or tocoferol and/or all-trans-vitamin A acid.

The present invention also comprises, in addition to the method according to the invention, a corresponding test kit with cells of at least one cell line, control samples of blood, blood serum or blood plasma, other body fluids or lavages or constituents thereof of a control subject without SIRS or sepsis and at least one reagent which responds to oxygen intermediates (ROI) and enters into a colour, light or other measurable reaction.

### EXAMPLES

The invention is now explained further with the help of non-limiting examples. In the examples, for comparison purposes, different commercially available cell lines were pre-treated for the test method according to the invention in sometimes different ways and then used in the method with blood plasma of sepsis patients.

### Example 1

### Test System HL-60 CH04

HL-60 cells (ATCC, CCL-240) were grown in accordance with the manufacturer's recommendations and then transferred into serum-free medium, e.g. Pro-CH04-CDM (Biowhittaker, 12-029Q) with 2 mM glutamax (Invitrogen, 35050-038). The cells were passaged every 2-3 days with a complete change of medium and an initial density of 0.5×10E6 cells/ml and kept under an atmosphere with 5% CO₂ at 37° C.

### Example 2

### Test System HL-60 VD3/IFN

HL-60 cells (ATCC, CCL-240) were grown in accordance with the manufacturer's recommendations and then passaged as described above in DMEM (Invitrogen, 11880-028) with 2 mM glutamax (Invitrogen, 35050-038) and 10% FBS (Invitrogen, 10099-141) and kept under an atmosphere with 5% CO₂ at 37° C. After 5 days' incubation with 1000 W interferon gamma (IFN) (Imukin, Boehringer Ingelheim) and 50 nM 1-alpha-25-dihydroxycholecalciferol (VD3) (Biomol, DM200-1000) with a change of medium after 3 days the adherent cells for use in the test were harvested.

### Example 3

### Test System THP-1 CH04

THP-1 cells (ATCC. TIP-202) were grown in accordance with the manufacturer's recommendations and then transferred into serum-free medium, e.g. Pro-CH04-CDM (Biowhittaker, 12-029Q) with 2 mM glutamax (Life Technologies, 35050-038). The cells were passaged every 2-3 days with a complete change of medium and an initial density of 0.4×10E6 cells/ml and kept under an atmosphere with 5% CO₂ at 37″C.

### Example 4

### Test System U-937 VD3/IFN

U-937 cells (ATCC, CRL-1593.2) were grown in accordance with the manufacturer's recommendations and then cultured in RPMI 1640 with glutamax (Invitrogen, 61870-010), 1 mM sodium pyruvate (Invitrogen, 11360-039) and 10% FBS (Invitrogen, 10099-141) (5% CO₂, 37° C., initial density 0.3×10E6 cells/ml, complete change of medium after 2-3 days). After 3 days' incubation with 1000 IU interferon gamma (IFN) (Imukin, Boehringer Ingelheim) and 50 nM 1-alpha-25-dihydroxycholecalciferol (VD3) (Biomol, DM200-1000) the cells for use in the test were harvested.

### Example 5

### Test Procedure

In each case the cells were centrifuged out from the medium at the end of the passage (250 xg, 5 min), then taken up in serum-free medium, e.g. Pro-CH04-CDM (Biowhittaker, 12-029Q) with 2 mM glutamax (Life Technologies, 35050-038) and set at a cell density of 10E6 cells/ml. The cells were kept for a maximum of 8 h at room temperature.

In each case 0.050 ml 0.65 mM lucigenin in PBS was added to 0.050 ml human serum in the cups of a white, non-transparent 96-well microtiter plate (e.g. Greiner, 655075). 0.050 ml PRS was introduced first instead of human serum for the zero-point determination without stimulating sample.

After 0.100 ml cell suspension (10E5 cells) in each case was added by pipetting, the chemiluminescence excitation of every sample was tracked with the help of a luminometer (e.g. Luminoskan RS, Labsystems) over a period of 45 minutes, wherein the integral of the measurement signals was recorded for 1 s every 3 min. For the evaluation, the sum of the single integrals was represented for every sample in each case as a percentage relative to the corresponding values of the controls (sera of healthy subjects).

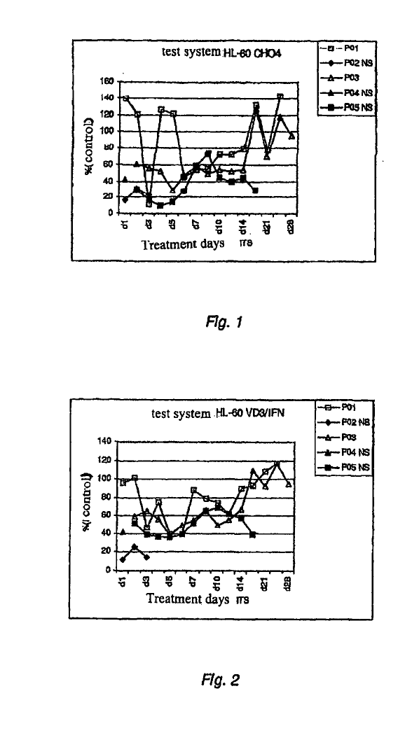

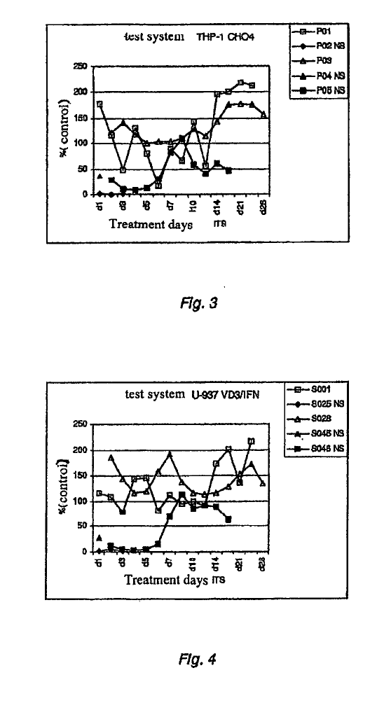

The results are presented in graphical form in FIGS. 1 to 4. They show respectively the comparison of the test systems HL-60 CH04 (FIG. 1), HL-60 VD3/IFN (FIG. 2), THP-1 CH04 (FIG. 3) and U-937 VD3/IFN (FIG. 4) for surviving (P01, P03) or non-surviving (NS) (P02, P04, P05) patients admitted with septic shock. The 100% value corresponds to the value that was obtained with serum of healthy subjects.

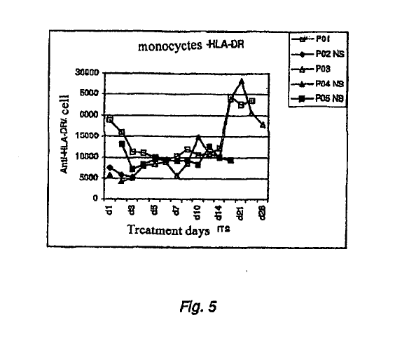

For comparison, in FIG. 5 the course of the immunosuppression is represented by means of the HLA-DR expression on monocytes, which was measured by means of a test customarily available in the trade (Quantibrite Anti-HLA-DR test kit, Becton-Dickinson 340827). In this comparison experiment, the same patient samples were used as in the tests according to FIGS. 1 to 4. A comparison of the results in FIGS. 1 to 4 with those of FIG. 5 clearly shows that the method according to the invention differentiates much better between the symptoms of patients with a high expectation of survival (P01, P03) and those with a contrasting high lethality risk (P02, P04, P05).

Referring to the results presented in FIGS. 1 to 4, the rise in the measured chemiluminescence signals in the case of the surviving patients P01 and P03 during the treatment clearly shows that recovery has set in, whereas patients P02, P04 and P05 died either much sooner (P02 and P04) or say on day 18 of the treatment (P05). There is a very clear surprising correlation of the strength of the chemiluminescence on the day of admission and shortly thereafter with the fact of whether the patients were able to recover due to the treatment or died despite the treatment (correlation between lethality and initial chemiluminescence signals). A relatively high chemiluminescence on the day of admission and shortly thereafter indicated a much higher probability of survival than a relatively low chemiluminescence at this time. The high chemiluminescence on the day of admission and shortly thereafter in the case of the surviving patients, which sometimes actually lay well above the measured values for healthy control patients, indicates a strong stimulation and excitation of the immune system of these patients. Such patients therefore have a good survival prognosis. In the case of the patients who died later, only a much smaller chemiluminescence reaction was recorded, indicating that these patients' immune system was already weakened to a greater or lesser extent on the day of admission.

This fact was surprising on the one hand and, on the other, because the lethality risk of a patient was diagnosable at a very early stage, opens up the possibility of taking further therapeutic measures such as those customary with such symptoms. In addition, the test according to the invention offers a very simple, comparatively cheap and above all rapid and reliable means of classifying the lethality risk of SIRS and sepsis patients. The reproducibility and comparability of the test becomes possible only through the use according to the invention of standardized and reproducible cell systems as biosensors.

### LITERATURE

Grimminger F et al.: Internist 38, 541-552 (1997)
Romaschin A D et al.: Sepsis 2, 119-125 (1998)
Oberhoffer M et al.: Clin Chem Lab Med 37(3) 363-368 (1999)
Mimoz et al.: Intensive Care Med 24, 185-188 (1998)
Al-Nawas B et al.: Eur J Med Res 1, 331-333 (1995/96)
Nussler A K et al.: Langenbeck's Arch Surg 384, 222-232 (1999)
Wakefield C H et al.: Brit J Surg 80, 205-209 (1993)
Asadullah K et al.: Crit. Care Med 23, 1976-1983 (1995)
Döcke W D et al.: Nature Med 3, 678-681 (1997)
Pascual et al.: Intensive Care Med 23, 743-748 (1997)
Breitman T R et al.: Proc Natl Acad Sci USA 77(5), 2936-2940 (1980)
Collins S J: Blood 70(5), 1233-1244 (1987)
Dong Y L et al.: J Trauma 34(3), 417-421 (1993)
Holzer K et al.: 5th World Congress on Trauma, Shock, Inflammation and Sepsis, Faist E ed. Monduzzi Editori publ., 593-597 (2000)
Bone R C et al.: Chest 101(6), 1644-1654
Knaus W A et al.: Crit. Care Med 13(10), 818-829

1. Method for the extracorporeal qualitative or semi-quantitative determination of the quantity of indicators for sirs or sepsis in the blood, blood serum, blood plasma, other body fluids or lavages or constituents thereof of human or animal subjects, wherein the method comprises the steps in which
a cell line is prepared in culture,
in at least one first preparation, cells of the cell line are brought into contact with blood, blood serum or blood plasma, other body fluids or lavages or constituents thereof of a human or animal subject to be examined, and with a reagent which responds to oxygen intermediates (ROI) and enters into a colour, light or other measurable reaction,
in at least one further preparation, cells of the cell line are brought into contact with blood, blood serum or blood plasma, other body fluids or lavages or constituents thereof of a human or animal control subject not ill with sirs or sepsis, and with a reagent which responds to oxygen intermediates (ROI) and enters into a colour, light or other measurable reaction,
the colour, light or other measurable reactions are measured in the preparations and the measured values of the subject to be examined are compared with those of the control subject.

2. Method according to claim 1, characterized in that the cell line is a leukocytary cell line or a leukocytes cell line.

3. Method according to one of claim 1 or 2, characterized in that the measurable reaction is a light reaction, preferably a chemiluminescence reaction.

4. Method according to one of claims 1 to 3, characterized in that the reagent is bis-N-methylacridiniumnitrate (lucigenin), 5-amino-1,2,3,4-tetrahydro-phthalazine-1,2-dione (luminol) or mixtures thereof, preferably lucigenin.

5. Method according to one of claim 3 or 4, characterized in that the chemiluminescence is measured by means of a luminometer, preferably by a kinesis over a period of 2-120 min.

6. Method according to claim 1 or 2, characterized in that the measurable reaction is a fluorescence reaction.

7. Method according to one of claim 1, 2 or 6, characterized in that the reagent is dihydrorhodamine, hydroethidium or a mixture of the two.

8. Method according to one of claim 1, 2, 6 or 7, characterized in that the fluorescence is measured by means of fluorometry (intracellular and extracellular) or throughflow cytometry (intracellular).

9. Method according to claim 1 or 2, characterized in that the measurable reaction is a colour reaction.

10. Method according to one of claim 1, 2 or 9, characterized in that the reagent is iron-III-cytochrome, nitrotetrazolium or a benzidine derivative, preferably 3,3′-5,5′-tetramethylbenzidine, or a mixture of at least two of the aforementioned.

11. Method according to one of claim 1, 2, 9 or 10, characterized in that the colour reaction is measured by means of spectral photometer.

12. Method according to one of the previous claims, characterized in that the cells of the cell line are HL-60 cells (ATCC, CCL-240), THP-1 cells (ATCC, THP-202) or U-937 cells (ATCC, CRL-1593.2).

13. Method according to one of the previous claims, characterized in that, before being brought into contact with the blood, blood plasma or blood serum, other body fluids or lavages or constituents thereof, the cells of the cell line are treated in culture for a period of time with inductors of the leukocyte differentiation, preferably with interferon gamma (IFN) and/or 1-alpha-25-dihydroxycholecalciferol (VD3) and/or granulocyte colony-stimulating factor (G-CSF) and/or tocoferol and/or all-trans-vitamin A acid.

14. Test kit with cells of at least one cell line, control samples of blood, blood serum or blood plasma, other body fluids or lavages or constituents thereof of a control subject without SIRS or sepsis and at least one reagent which responds to oxygen intermediates (ROI) and enters into a colour, light or other measurable reaction, according to one of claims 1 to 13.
